SEQUENCE LISTING (6 sequences)

SEQ ID NO: 1 - DNA, 3500 bp, Clostridium histolyticum
aactcctcccgttttaaatagaatctttataaatttattttatcctaatattctcttatatacttaattaaatattaataaaaaattaatgaacaggtatatcttaacaaaaattaaacaaaaattaaacaaatatataacaaatattaataaataatgttgacactactaaaaaatggcgttatactttaataaaaggcttatataattcctcaatacaaatattcagataattatgaaaagagcataaatgaaggaattatgaattttttaaaaattattttaaatagggggaagactatgaaaaggaaatgtttatctaaaaggcttatgttagctataacaatggctacaatatttacagtgaacagtacattaccaatttatgcagctgtagataaaaataatgcaacagcagctgtacaaaatgaaagtaagaggtatacagtatcatatttaaagactttaaattattatgacttagtagatttgcttgttaagactgaaattgagaatttaccagacctttttcagtatagttcagatgcaaaagagttctatggaaataaaactcgtatgagctttatcatggatgaaattggtagaagggcacctcagtatacagagatagatcataaaggtattcctactttagtagaagttgtaagagctggattttacttaggattccataacaaggaattgaatgaaataaacaagaggtcttttaaagaaagggtaataccttctatattagcaattcaaaaaaatcctaattttaaactaggtactgaagttcaagataaaatagtatctgcaacaggacttttagctggtaatgaaacagcgcctccagaagttgtaaataattttacaccaatacttcaagactgtataaagaatatagacagatacgctcttgatgatttaaagtcaaaagcattatttaatgttttagctgcacctacctatgatataactgagtatttaagagctactaaagaaaaaccagaaaacactccttggtatggtaaaatagatgggtttataaatgaacttaaaaagttagctctttatggaaaaataaatgataataactcttggataatagataacggtatatatcatatagcacctttagggaagttacatagcaataataaaataggaatagaaactttaacagaggttatgaaagtttatccttatttaagtatgcaacatttacaatcagcagatcaaattaagcgtcattatgattcaaaagatgctgaaggaaacaaaatacctttagataagtttaaaaaggaaggaaaagaaaaatactgtccaaaaacttatacatttgatgatggaaaagtaataataaaagctggtgctagagtagaagaagaaaaagttaaaagactatactgggcatcaaaggaagttaactctcaattctttagagtatacggaatagacaaaccattagaagaaggtaatccagatgatatattaacaatggttatctacaacagtcccgaagaatataaactcaatagtgttctatacggatatgatactaataatggtggtatgtatatagagccagaaggaactttcttcacctatgaaagagaagctcaagaaagcacatacacattagaagaattatttagacatgaatatacacattatttgcaaggaagatatgcagttccaggacaatggggaagaacaaaactttatgacaatgatagattaacttggtatgaagaaggtggagcagaattatttgcaggttctactagaacttctggaatattaccaagaaagagtatagtatcaaatattcataatacaacaagaaataatagatataagctttcagacactgtacattctaaatatggtgctagttttgaattctataattatgcatgtatgtttatggattatatgtataataaagatatgggtatattaaataaactaaatgatcttgcaaaaaataatgatgttgatggatatgataattatattagagatttaagttctaattatgctttaaatgataaatatcaagatcatatgcaggagcgcatagataattatgaaaatttaacagtgccttttgtagctgatgattatttagtaaggcatgcttataagaaccctaatgaaatttattctgaaatatctgaagtagcaaaattaaaggatgctaagagtgaagttaagaaatcacaatattttagtacctttactttgagaggtagttacacaggtggagcatctaaggggaaattagaagatcaaaaagcaatgaataagtttatagatgattcacttaagaaattagatacgtattcttggagtgggtataaaactttaactgcttatttcactaattataaagttgactcttcaaatagagttacttatgatgtagtattccacggatatttaccaaacgaaggtgattccaaaaattcattaccttatggcaagatcaatggaacttacaagggaacagagaaagaaaaaatcaaattctctagtgaaggctctttcgatccagatggtaaaatagtttcttatgaatgggatttcggagatggtaataagagtaatgaggaaaatccagagcattcatatgacaaggtaggaacttatacagtgaaattaaaagttactgatgacaagggagaatcttcagtatctactactactgcagaaataaaggatctttcagaaaataaacttccagttatatatatgcatgtacctaaatccggagccttaaatcaaaaagttgttttctatggaaaaggaacatatgacccagatggatctatcgcaggatatcaatgggactttggtgatggaagtgattttagcagtgaacaaaacccaagccatgtatatactaaaaaaggtgaatatactgtaacattaagagtaatggatagtagtggacaaatgagtgaaaaaactatgaagattaagattacagatccggtatatccaataggcactgaaaaagaaccaaataacagtaaagaaactgcaagtggtccaatagtaccaggtatacctgttagtggaaccatagaaaatacaagtgatcaagattatttctattttgatgttataacaccaggagaagtaaaaatagatataaataaattagggtacggaggagctacttgggtagtatatgatgaaaataataatgcagtatcttatgccactgatgatgggcaaaatttaagtggaaagtttaaggcagataaaccaggtagatattacatccatctttacatgtttaatggtagttatatgccatatagaattaatatagaaggttcagtaggaagataatattttattagttgaggtaactccatataatagcttagctatttcttatggagttactttttatatgtaataaaattttgacttaaattatgattttttgctataatggtttggaaattaatgatttataattt

SEQ ID NO: 2 - DNA, 6774 bp, Homo sapiens
cggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgattttaatggccacatctaatctcatttcacatgaaagaagaagtatattttagaaatttgttaatgagagtaaaagaaaataaatgtgtatagctcagtttggataattggtcaaacaattttttatccagtagtaaaatatgtaaccattgtcccagtaaagaaaaataacaaaagttgtaaaatgtatattctcccttttatattgcatctgctgttacccagtgaagcttacctagagcaatgatctttttcacgcatttgctttattcgaaaagaggcttttaaaatgtgcatgtttagaaacaaaatttcttcatggaaatcatatacattagaaaatcacagtcagatgtttaatcaatccaaaatgtccactatttcttatgtcattcgttagtctacatgtttctaaacatataaatgtgaatttaatcaattcctttcatagttttataattctctggcagttccttatgatagagtttataaaacagtcctgtgtaaactgctggaagttcttccacagtcaggtcaattttgtcaaacccttctctgtacccatacagcagcagcctagcaactctgctggtgatgggagttgtattttcagtcttcgccaggtcattgagatccatccactcacatcttaagcattcttcctggcaaaaatttatggtgaatgaatatggctttaggcggcagatgatatacatatctgacttcccaaaagctccaggatttgtgtgctgttgccgaatactcaggacggacctgaattctgattttataccagtctcttcaaaaacttctcgaaccgctgtgtctcctacgtaaaaaaagagatgtacaaatcaataataattacacttttagaaactgtatcatcaaagattttcagttaaagtagcattatgtaaaggctcaaaacattaccctaacaaagtaaagttttcaatacaaattctttgccttgtggatatcaagaaatcccaaaatattttcttaccactgtaaattcaagaagcttttgaaatgctgaatatttctttggctgctacttggaggcttatctacctgtacatttttggggtcagctctttttaacttcttgctgctctttttcccaaaaggtaaaaatatagattgaaaagttaaaacattttgcatggctgcagttcctttgtttcttgagataagattccaaagaacttagattcatttcttcaacaccgaaatgctggaggtgtttgatcagttttcaagaaacttggaatataaataattttataattcaacaaaggttttcacattttataaggttgatttttcaattaaatgcaaatttgtgtggcaggatttttattgccattaacatatttttgtggctgctttttctacacatccagatggtccctctaactgggctttctctaattttgtgatgttctgtcattgtctcccaaagtatttaggagaagccctttaaaaagctgccttcctctaccactttgctggaaagcttcacaattgtcacagacaaagatttttgttccaatactcgttttgcctctatttttcttgtttgtcaaatagtaaatgatatttgcccttgcagtaattctactggtgaaaaacatgcaaagaagaggaagtcacagaaacatgtctcaattcccatgtgctgtgactgtagactgtcttaccatagactgtcttacccatcccctggatatgctcttgttttttccctctaatagctatggaaagatgcatagaaagagtataatgttttaaaacataaggcattcgtctgccatttttcaattacatgctgacttcccttacaattgagatttgcccataggttaaacatggttagaaacaactgaaagcataaaagaaaaatctaggccgggtgcagtggctcatgcctatattccctgcactttgggaggccaaagcaggaggatcgcttgagcccaggagttcaagaccaacctggtgaaaccccgtctctacaaaaaaacacaaaaaatagccaggcatggtggcgtgtacatgtggtctcagatacttgggaggctgaggtgggagggttgatcacttgaggctgagaggtcaaggttgcagtgagccataatcgtgccactgcagtccagcctaggcaacagagtgagactttgtctcaaaaaaagagaaattttccttaataagaaaagtaatttttactctgatgtgcaatacatttgttattaaatttattatttaagatggtagcactagtcttaaattgtataaaatatcccctaacatgtttaaatgtccatttttattcattatgctttgaaaaataattatggggaaatacatgtttgttattaaatttattattaaagatagtagcactagtcttaaatttgatataacatctcctaacttgtttaaatgtccatttttattctttatgtttgaaaataaattatggggatcctatttagctcttagtaccactaatcaaaagttcggcatgtagctcatgatctatgctgtttctatgtcgtggaagcaccggatgggggtagtgagcaaatctgccctgctcagcagtcaccatagcagctgactgaaaatcagcactgcctgagtagttttgatcagtttaacttgaatcactaactgactgaaaattgaatgggcaaataagtgcttttgtctccagagtatgcgggagacccttccacctcaagatggatatttcttccccaaggatttcaagatgaattgaaatttttaatcaagatagtgtgctttattctgttgtattttttattattttaatatactgtaagccaaactgaaataacatttgctgttttataggtttgaagaacataggaaaaactaagaggttttgtttttatttttgctgatgaagagatatgtttaaatatgttgtattgttttgtttagttacaggacaataatgaaatggagtttatatttgttatttctattttgttatatttaataatagaattagattgaaataaaatataatgggaaataatctgcagaatgtgggttttcctggtgtttccctctgactctagtgcactgatgatctctgataaggctcagctgctttatagttctctggctaatgcagcagatactcttcctgccagtggtaatacgattttttaagaaggcagtttgtcaattttaatcttgtggatacctttatactcttagggtattattttatacaaaagccttgaggattgcattctattttctatatgaccctcttgatatttaaaaaacactatggataacaattcttcatttacctagtattatgaaagaatgaaggagttcaaacaaatgtgtttcccagttaactagggtttactgtttgagccaatataaatgtttaactgtttgtgatggcagtattcctaaagtacattgcatgttttcctaaatacagagtttaaataatttcagtaattcttagatgattcagcttcatcattaagaatatcttttgttttatgttgagttagaaatgccttcatatagacatagtctttcagacctctactgtcagttttcatttctagctgctttcagggttttatgaattttcaggcaaagctttaatttatactaagcttaggaagtatggctaatgccaacggcagtttttttcttcttaattccacatgactgaggcatatatgatctctgggtaggtgagttgttgtgacaaccacaagcactttttttttttttaaagaaaaaaaggtagtgaatttttaatcatctggactttaagaaggattctggagtatacttaggcctgaaattatatatatttggcttggaaatgtgtttttcttcaattacatctacaagtaagtacagctgaaattcagaggacccataagagttcacatgaaaaaaatcaatttatttgaaaaggcaagatgcaggagagaggaagccttgcaaacctgcagactgctttttgcccaatatagattgggtaaggctgcaaaacataagcttaattagctcacatgctctgctctcacgtggcaccagtggatagtgtgagagaattaggctgtagaacaaatggccttctctttcagcattcacaccactacaaaatcatcttttatatcaacagaagaataagcataaactaagcaaaaggtcaataagtacctgaaaccaagattggctagagatatatcttaatgcaatccattttctgatggattgttacgagttggctatataatgtatgtatggtattttgatttgtgtaaaagttttaaaaatcaagctttaagtacatggacatttttaaataaaatatttaaagacaatttagaaaattgccttaatatcattgttggctaaatagaataggggacatgcatattaaggaaaaggtcatggagaaataatattggtatcaaacaaatacattgatttgtcatgatacacattgaatttgatccaatagtttaaggaataggtaggaaaatttggtttctatttttcgatttcctgtaaatcagtgacataaataattcttagcttattttatatttccttgtcttaaatactgagctcagtaagttgtgttaggggattatttctcagttgagactttcttatatgacattttactatgttttgacttcctgactattaaaaataaatagtagatacaattttcataaagtgaagaattatataatcactgctttataactgactttattatatttatttcaaagttcatttaaaggctactattcatcctctgtgatggaatggtcaggaatttgttttctcatagtttaattccaacaacaatattagtcgtatccaaaataacctttaatgctaaactttactgatgtatatccaaagcttctcattttcagacagattaatccagaagcagtcataaacagaagaataggtggtatgttcctaatgatattatttctactaatggaataaactgtaatattagaaattatgctgctaattatatcagctctgaggtaatttctgaaatgttcagactcagtcggaacaaattggaaaatttaaatttttattcttagctataaagcaagaaagtaaacacattaatttcctcaacatttttaagccaattaaaaatataaaagatacacaccaatatcttcttcaggctctgacaggcctcctggaaacttccacatatttttcaactgcagtataaagtcagaaaataaagttaacataactttcactaacacacacatatgtagatttcacaaaatccacctataattggtcaaagtggttgagaatatattttttagtaattgcatgcaaaatttttctagcttccatcctttctccctcgtttcttctttttttgggggagctggtaactgatgaaatcttttcccaccttttctcttcaggaaatataagtggttttgtttggttaacgtgatacattctgtatgaatgaaacattggagggaaacatctactgaatttctgtaatttaaaatattttgctgctagttaactatgaacagatagaagaatcttacagatgctgctataaataagtagaaaatataaatttcatcactaaaatatgctattttaaaatctatttcctatattgtatttctaatcagatgtattactcttattatttctattgtatgtgttaatgattttatgtaaaaatgtaattgcttttcatgagtagtatgaataaaattgattagtttgtgttttcttgtctccc

SEQ ID NO: 3 - protein, 599 aa, Artificial Sequence
  GST-bFGF-PKD-CBD
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKSGIPEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 4 - DNA, 1800 bp, Artificial Sequence
  GST-bFGF-PKD-CBD
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatctatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagcggaattcccgaaataaaggatctttcagaaaataaacttccagttatatatatgcatgtacctaaatccggagccttaaatcaaaaagttgttttctatggaaaaggaacatatgacccagatggatctatcgcaggatatcaatgggactttggtgatggaagtgattttagcagtgaacaaaacccaagccatgtatatactaaaaaaggtgaatatactgtaacattaagagtaatggatagtagtggacaaatgagtgaaaaaactatgaagattaagattacagatccggtatatccaataggcactgaaaaagaaccaaataacagtaaagaaactgcaagtggtccaatagtaccaggtatacctgttagtggaaccatagaaaatacaagtgatcaagattatttctattttgatgttataacaccaggagaagtaaaaatagatataaataaattagggtacggaggagctacttgggtagtatatgatgaaaataataatgcagtatcttatgccactgatgatgggcaaaatttaagtggaaagtttaaggcagataaaccaggtagatattacatccatctttacatgtttaatggtagttatatgccatatagaattaatatagaaggttcagtaggaagataa

SEQ ID NO: 5 - protein, 375 aa, Artificial Sequence
  bFGF-PKD-CBD
GSMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKSGIPEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 6 - DNA, 1128 bp, Artificial Sequence
  bFGF-PKD-CBD
ggatctatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagcggaattcccgaaataaaggatctttcagaaaataaacttccagttatatatatgcatgtacctaaatccggagccttaaatcaaaaagttgttttctatggaaaaggaacatatgacccagatggatctatcgcaggatatcaatgggactttggtgatggaagtgattttagcagtgaacaaaacccaagccatgtatatactaaaaaaggtgaatatactgtaacattaagagtaatggatagtagtggacaaatgagtgaaaaaactatgaagattaagattacagatccggtatatccaataggcactgaaaaagaaccaaataacagtaaagaaactgcaagtggtccaatagtaccaggtatacctgttagtggaaccatagaaaatacaagtgatcaagattatttctattttgatgttataacaccaggagaagtaaaaatagatataaataaattagggtacggaggagctacttgggtagtatatgatgaaaataataatgcagtatcttatgccactgatgatgggcaaaatttaagtggaaagtttaaggcagataaaccaggtagatattacatccatctttacatgtttaatggtagttatatgccatatagaattaatatagaaggttcagtaggaagataa

(19) United States
(12) Patent Application Publication
(10) Pub. No.: US 2018/0140742 A1
(43) Pub. Date: May 24, 2018

(54) GRAFT MATERIAL FOR NERVE REGENERATION, METHOD FOR PRODUCING GRAFT MATERIAL FOR NERVE REGENERATION, AND KIT FOR PRODUCING GRAFT MATERIAL FOR NERVE REGENERATION

(71) Applicants: SCHOOL JURIDICAL PERSON KITASATO INSTITUTE, Tokyo (JP); ATREE, INC., Tokyo (JP); NATIONAL UNIVERSITY CORPORATION OKAYAMA UNIVERSITY, Okayama-shi, Okayama (JP); NATIONAL UNIVERSITY CORPORATION KAGAWA UNIVERSITY, Takamatsu-shi, Kanagawa (JP); NIPPI, INCORPORATED, Tokyo (JP)

(72) Inventors: Kentaro UCHIDA, Sagamihara-shi (JP); Gen INOUE, Sagamihara-shi (JP); Hisako FUJIMAKI, Sagamihara-shi (JP); Masashi TAKASO, Sagamihara-shi (JP); Taro SAKU, Yamatokooriyama-shi (JP); Yoshihiro ISOBE, Yamatokooriyama-shi (JP); Osamu MATSUSHITA, Okayama-shi (JP); Takehiko MIMA, Okayama-shi (JP); Nozomu NISHI, Kita-gun (JP); Shunji HATTORI, Toride-shi (JP); Keisuke TANAKA, Toride-shi (JP); Takayuki OGURA, Toride-shi (JP)

(21) Appl. No.: 15/519,283

(22) PCT Filed: Oct. 16, 2015
(86) PCT No.: PCT/JP2015/079334

§ 371 Date: Apr. 14, 2017

(30) Foreign Application Priority Data
Oct. 16, 2014 (JP) 2014-212085

(51) Int. Cl. A61L 27/36; A61F 2/00; A61L 27/24; A61L 27/22

(52) U.S. Cl. CPC A61L 27/3675; A61F 2/0077; A61L 27/24; A61L 27/3695; C12N 2501/115; A61L 27/227; A61L 2430/32; A61F 2002/0068; A61L 27/3604

(57) ABSTRACT

1) A graft material for nerve regeneration characterizing by comprising collagen-based materials containing collagen having an orientation. 2) A method for producing a graft material for nerve regeneration comprising a step of immersing the collagen-based materials containing collagen having an orientation in a solution containing a collagen-binding site-containing growth factor comprising a receptor agonist peptide and a collagen-binding peptide and binding the collagen-binding site-containing growth factor to the collagen. 3) A kit for producing a graft material for nerve regeneration characterized by comprising collagen-based materials containing collagen having an orientation, and a collagen-binding site-containing growth factor comprising a receptor agonist peptide and a collagen-binding peptide.

### TECHNICAL FIELD

The present invention relates to a graft material for nerve regeneration, a method for producing a graft material for nerve regeneration, and a kit for producing a graft material for nerve regeneration.

Priority is claimed on Japanese Patent Application No. 2014-212085, filed Oct. 16, 2014, the content of which is incorporated herein by reference.

### BACKGROUND ART

Autologous nerve graft for transplanting healthy nerve tissue with respect to nerve damage caused by a traumatic car accident or tumor ectomy has been performed. However, there are limitations to the length and diameter of nerve tissue that can be used as a donor and the problem of damage to the donor site. Recently, although artificial nerve comprising biomaterial has been developed, sufficient results have not been obtained.

On the other hands, the promotion of self-repair of nerve damage has also been attempted conventionally. In Patent Literature 1, a nerve regeneration-inducing tube is disclosed using collagen as a scaffold for nerve regeneration. In Patent Literatures 2 and 3, a nerve regeneration-inducing tube in which a tubular body knitted with biodegradable polymer fibers is coated and filled with collagen is disclosed.

Further, in recent years, collagen having an orientation, which can be used as a biograft material, has been developed (Patent Literatures 4 and 5). In Patent Literature 5, as a use of collagen having an orientation as a biocompatible material, a method of producing an oriented collagen/apatite material in which apatite having an orientation similar to or almost the same as the direction of orientation of the collagen is produced and fixed on a surface and/or inside of the collagen by seeding osteoblasts or mesenchymal stem cells is disclosed. This is to provide a biocompatible material having characteristics similar to bone tissue.

Nerve defects cause a considerable decrease in the quality of life of a patient having a severe dysfunction over time, and long-term treatment is directly connected to the delayed social reintegration of a patient and increased medical expenses. Therefore, for example, the development of technology capable of restoring nerve damage earlier is required.

### CITATION LIST

### Patent Literature

### [Patent Literature 1]

Japanese Patent No. 4572996

### [Patent Literature 2]

Japanese Patent No. 4596335

### [Patent Literature 3]

Japanese Patent No. 4640533

### [Patent Literature 4]

PCT International Publication No. WO2012/114707

### [Patent Literature 5]

Japanese Unexamined Patent Application, First Publication No. 2012-65742

### SUMMARY OF INVENTION

### Technical Problem

The present invention was accomplished in consideration of such circumstances, and is directed to providing a graft material for nerve regeneration capable of effectively regenerating nerves.

### Solution to Problem

As a result of extensive research to solve the above-mentioned problem, the inventors have found that by using collagen-based materials comprising collagen having an orientation, which has not been conventionally used for nerve regeneration, as a graft material for nerve regeneration, effective regeneration of a damaged region of a nerve can be realized, and thus completed the present invention. That is, the present invention is as follows.

(1) A graft material for nerve regeneration comprising collagen-based materials containing collagen having an orientation.

(2) The graft material for nerve regeneration described in (1), wherein a collagen-binding site-containing growth factor comprising a receptor agonist peptide and a collagen-binding peptide bind to the collagen.

(3) The graft material for nerve regeneration described in (1) or (2), having a hollow cylindrical shape and in which at least a part of the inner surface of the cylinder is constituted of the collagen-based materials.

(4) The graft material for nerve regeneration described in (3), wherein the collagen has an orientation in a direction in which openings at both ends of the cylinder are connected.

(5) The graft material for nerve regeneration described in any one of (2) to (4),

wherein the collagen-binding site-containing growth factor peptide and the collagen-binding peptide are bound via a linker, and

the linker is a polycystic kidney I domain of collagenase.

(6) The graft material for nerve regeneration described in any one of (2) to (5), wherein the growth factor receptor agonist peptide is a basic fibroblast growth factor.

(7) The graft material for nerve regeneration described in any one of (1) to (6), wherein the collagen-based materials are comprised of a plurality of collagen-based materials layers.

(8) The graft material for nerve regeneration described in any one of (1) to (7), wherein a thickness of the collagen-based materials is 50 μm or more and 200 μm or less.

(9) A method for producing a graft material for nerve regeneration, comprising a step of immersing collagen-based materials containing collagen having an orientation in a solution containing a collagen-binding site-containing growth factor comprising a receptor agonist peptide and a collagen-binding peptide, and binding the collagen-binding site-containing growth factor to the collagen.

(10) A kit for producing a graft material for nerve regeneration, comprising collagen-based materials containing collagen having an orientation, and a collagen-binding site-containing growth factor comprising a receptor agonist peptide and a collagen-binding peptide.

### Advantageous Effects of Invention

According to the present invention, a graft material for nerve regeneration having superior in nerve regeneration efficiency can be provided.

### BRIEF DESCRIPTION OF DRAWINGS

FIG. 1 is a photograph showing the outside of an oriented collagen tube A manufactured in Examples.

FIG. 2 is (a) a macroscopic finding (photograph) of an oriented collagen tube-grafted site 12 weeks after the transplantation, (b) is an HE-stained image of the oriented collagen tube-grafted site 12 weeks after the transplantation, and (c) is an enlarged image of the HE-stained image shown in FIG. 2(b).

FIG. 3 is a Fast Blue-stained image for ganglion cells.

FIG. 4 is a graph showing an evaluation result of a motor function for rats after transplantation of an oriented collagen tube.

FIG. 5 is a graph showing a relationship between amounts of a bFGF-PKD-CBD fusion protein added into a solution and amounts of a bFGF-PKD-CBD fusion protein binding to an oriented collagen tube.

FIG. 6 is a graph showing a behavioral evaluation result using a von Frey filament to rats after transplantation of an oriented collagen tube.

FIG. 7 is a toluidine blue-stained image of a nerve regenerated in a collagen tube-grafted site.

FIG. 8 is a schematic diagram of the molecular phylogenetic tree of bacterial collagenase having CBD and a domain thereof.

### DESCRIPTION OF EMBODIMENTS

<<Graft Material for Nerve Regeneration>>

<Collagen-Based Materials>

A graft material for nerve regeneration of the present invention comprises collagen-based materials containing collagen having an orientation.

The collagen having an orientation means a collagen in which a travel direction of a fibrous collagen such as a single collagen gel or dry collagen gel uniforms in some direction. In the case that the collagen having an orientation is coated on a substrate comprises a metal, a ceramic, a polymer material or a biomaterial (hereinafter it is also referred to as a collagen substrate), the collagen having an orientation means a collagen in which a travel direction of a fibrous collagen such as collagen gel or dry collagen gel on a substrate such as a metal, a ceramic, a polymer material or a biomaterial formed to various shapes, uniforms in some direction.

The travel direction of fibrous collagen uniformed in some direction means that a state in which a ratio of fibrous collagen in which a travel direction uniforms in some direction is higher than a ratio of fibrous collagen in which a travel direction uniforms in different direction in collagen-based materials.

Meanwhile, the term “travel direction” of the fibrous collagen has the same meaning as a direction of an orientation, a direction, an orientation, an orientation property, and a direction of an orientation property.

A method for preparing collagen gel having an orientation is not particularly limited, but according to a common procedure. For example, as a method of imparting an orientation to a collagen gel at a larger size than a millimeter order, a method for giving a flow of a fixed direction to a during a process of gelation of a collagen solution is suggested, but other methods may be used. As other methods, mention may be made of a method for applying a strong magnetic field during a process of forming a collagen fiber, a method of spin-coating a collagen gel, and a method of drawing a collagen gel to predetermined direction mechanically (and physically).

In the case that a collagen gel fragment having an orientation is prepared by the a method of applying a strong magnetic field during a process of forming a collagen fiber, since the collagen fiber is set in array in perpendicular direction to the magnetic field, if it is kept to apply a magnetic field from the same direction, it is possible to obtain a two-dimensional orientation, and if it is kept to apply a rotational magnetic field, it is possible to obtain a single axis orientation. It is possible to use a method of applying a magnetic field if such collagen gel having orientation is used as a starting material. However, if the magnetic field is used, it is possible to produce only those of the collagen gel having uniform orientation, and a macro shape also tends to be limited. On the other hand, in the case of that a collagen gel having an orientation is prepared by the method of giving a flow of fixed direction to a collagen solution during a process of gelating a collagen solution, it is possible to produce a collagen having three-dimensional orientation by forming and laminating a various shape including a sheet-like shape because of the use of a flow of liquid.

In such a method, an oriented collagen (a single collagen) can be obtained by using a flow of a collagen solution to give an orientation during a process of solidifying the collagen solution to collagen gel. According to the method, it is possible to produce an orientated collagen or a collagen gel fragment with various shapes (line, plane, solid) such as a string shape, a ribbon-shape with a large width. Further, during a process, a control a velocity of a flow also makes it possible to control of the orientation. Therefore, it is possible to control a direction of the orientation or a degree of the orientation thereby giving a desired distribution even if it is in the same collagen gel.

For example, an explanation as to a method of giving a flow in a fixed direction to the collagen solution during a process of gelating a collagen solution is as follows. Although a concentration of the collagen solution is preferably 10 mg/ml or more from a viewpoint that an obtained collagen or a collagen substrate can have an enough mechanical strength, it may be about 3 mg/ml or more. An origin of a collagen is not limited. Further, a seed, a site of a tissue, an age etc., of an animal derived from are not particularly limited. For example, it is possible to use one derived from animal origin such as a rat tail, pig hide, cow skin, ostrich or fish. That is, it is possible to use a collagen obtained from skin, bone, cartilage, tendon or an internal organ of a mammal (for example, a cattle, pig, horse, rabbit or mouse etc.) or a bird (for example, a chicken etc.). A collagen-like protein derived from skin, bone, cartilage, fin, scale or an internal organ of fishes (for example, pacific cod, paralichthys olivaceus, flatfish, salmon, trout, tuna, chub mackerel, sea bream, sardine or shark etc.) may also be used. Moreover, a method of extracting collagen is not particularly limited, but a common method of extracting may be used. Further, collagen obtained by a gene recombination technique, other than from the extraction from animal tissue may be used. Further, in order to suppress antigenicity, an enzyme-treated atelocollagen may be used. Further, as a collagen, unmodified soluble collagen such as acid-soluble collagen, neutral salt-soluble collagen, enzyme-soluble collagen (atelocollagen), chemically-modified collagen such as acylation such as succinylation or phthalization, esterification such as methylation, deamination of alkali solubilization or, and further an insoluble collagen such as tendon collagen etc. may be used. Further, a chemical cross-linking agent, a medicinal agent or an air bubble such as oxygen may also be introduced into a collagen solution. A method of introducing them is not particularly limited, but according to a common procedure.

It is possible to quantitatively assess direction of the orientation or degree of orientation as an obtained collagen by using, for example, raman spectroscopy microscope. A raman spectroscopy is to examine a component which a frequency modulation of scattered light caused by hitting against molecular is occurred according to molecular vibration, by means of the use of a spectroscopy, and thereby making it possible to obtain information as to composition of a target for analysis or a crystal structure to analysis an orientation of collagen.

Since the graft material for nerve regeneration of the present invention comprises collagen-based materials containing collagen having an orientation, it is possible to stimulate to a regeneration of nerve cells and nerve tissue on the line with an orientation of collagen. Here, it can be considered that the collagen-based materials play a role as a scaffold for nerve cells. Since it is also important for nerve regeneration to regenerate a spatial arrangement, use of collagen-based materials containing collagen having an orientation is very useful. In the case of repairing nerve damage, it is possible to regenerate nerve efficiently, for example, by using the method that the graft material for nerve regeneration is placed on a nerve cut site, and collagen orientation is aligned with a direction in which an original nerve passes.

(Shape)

A shape of the graft material for nerve regeneration of the present invention is not particularly limited, but may be a shape such as a ribbon, sheet, tube, sponge, grain (particle), rod, ring, spiral, spring, disc, dome or block etc.

In order to prepare the shape of a graft material mentioned above, for example, it is possible to prepare a sheet type of collagen material (fragment) from a string shape, and to further process the collagen material to make a many sort of final shape type of the three-dimensional collagen material. The three-dimensional collagen material can be prepared by the method disclosed in PCT International Publication No. WO2012/114707.

The graft material for nerve regeneration of the present invention preferably has a hollow cylindrical shape (tube shape) among the above-exemplified shapes. Furthermore, at least a part or all of the inner surface of the cylinder is preferably comprised of the above-described collagen-based materials. In addition, at least a part or all of the inner surface of the cylinder is preferably constituted of the above-described collagen-based materials. In a graft material for nerve regeneration having a cylinder shape, a nerve can be regenerated in inside the cylinder. The cylinder can prevent neighboring tissue from intruding into the cylinder, and also can preserve nerves inside the cylinder so as to regenerate nerves more effectively.

In the case that a graft material for nerve regeneration has a hollow cylindrical shape, the above-described collagen preferably has an orientation in a direction in which openings of both ends of the above-described cylinder are connected. Since the direction in which the openings of both ends of cylinder are connected become a direction in which a defective nerves are connected at their ends, for example, when the cylinder-shaped graft material for nerve regeneration is inserted into the defective part of nerve, a nerve can be regenerated more effectively.

As the case that a graft material for nerve regeneration has a hollow cylindrical shape, a case of collagen-based materials themselves have a cylinder shape may be mentioned. In this case, the collagen-based materials are preferably a seamless tube without a junction. The junction is a connection part between ends of plate-shaped collagen-based materials, which is formed when they are connected each other to form cylinder-shape. A seamless tube is preferable because in the seamless tube, cell can be more smoothly grown on the inner surface of the tube.

The graft material for nerve regeneration of the present invention preferably comprises a biodegradable material and has collagen-based materials containing collagen having an orientation. Since the graft material for nerve regeneration comprising the biodegradable material is degraded after the completion of nerve regeneration in a living organism that is subjected to transplantation, a burden on the recipient after the transplantation can be reduced.

Further, the above-described collagen-based materials having the graft material for nerve regeneration of the present invention may be comprised of a plurality of collagen-based materials layers. It is possible to adjust a physical property of the collagen-based materials such as a thickness or strength easily by stacking multiple layers of the collagen-based materials. Therefore, without using other supports, the physical property of the graft material for nerve regeneration can be adjusted only with adjustment of biodegradable collagen-based materials.

As examples of an embodiment of the graft material for nerve regeneration include a graft material having a hollow cylindrical shape in which at least a part of the inner surface of the cylinder is constituted of the collagen-based materials and the collagen has an orientation in a direction in which the openings of both ends of the cylinder are connected and the collagen-based materials comprise a plurality of collagen-based materials layers. Here, the collagen of the collagen-based materials layer on the innermost surface of the cylinder preferably has an orientation in a direction in which the openings of both ends of the cylinder are connected. Collagen layers other than an innermost collagen-based materials layer surface may or may not have an orientation. In the case that collagen layers other than the innermost collagen-based materials layer has an orientation, the direction of the orientation of the collagen layer other than the innermost collagen-based materials layer is not particularly limited. However, when it is considered that the innermost layer is biodegraded and a layer other than the innermost collagen-based materials layer is exposed to the inner surface of the cylinder, collagen layer other than the innermost collagen-based materials layer also preferably has the orientation in a direction in which the openings of both ends of the above-described cylinder are connected. On the other hand, when it is considered to an increase in strength such as sewability, the collagen layer other than the innermost collagen-based materials layer preferably has the orientation in a direction in which the openings of both ends of the above-described cylinder are connected.

In such manner, it is possible to increase functionality of the graft material for nerve regeneration by the collagen-based materials being multilayered.

A thickness of the collagen-based materials is preferably 50 μm or more, more preferably 70 μm or more.

The thickness of the collagen-based materials is preferably 200 μm or less, more preferably 170 μm or less, further more preferably 130 μm or less.

The thickness of the collagen-based materials is preferably 50 μm or more to 200 μm or less, more preferably 70 μm or more to 170 μm or less, further more preferably 70 μm or more to 130 μm or less. Generally, collagen-based materials having a thickness of 50 μm or more facilitates transplantation. Further, generally, collagen-based materials having a thickness of 200 μm or less are preferable because the time required for biodegradation is not too long and thus the burden on the living body is reduced.

The thickness of the collagen-based materials can be obtained by measuring the thicknesses of approximately 10 randomly selected spots of the dried collagen-based materials and obtaining the averaged value.

In the case of the collagen-based materials being multilayered, the thickness of the collagen-based materials refers to a thickness of the entire multilayered layers. In the case of the collagen-based materials being multilayered, examples of the thickness of a single layer include approximately 10 to 15 μm.

In the present invention, it is possible to basically produce a dry type of the collagen-based materials. However, it is also possible to produce a gel type of the collagen-based materials obtained by immersing a dry type of collagen-based materials into PBS or the like.

In the specification, the dry type of collagen-based materials means collagen-based materials containing water of 0 to 30-mass %. The water content can be measured by a normal pressure heating drying method.

In general, although a part of tissue of the collagen-based materials may be destroyed and removed if it is dried, the dry type of collagen-based materials will be easy to use from a view point of storage stability (it is easy to maintain a shape. A gel is easy to corrupt since it contains water), and transit performance (A gel is easy to destroy since it contains water. It may become deformed when it is taken up from a vessel).

In the present invention, the dry type of collagen-based materials can be used as a gel type of the collagen-based materials by setting back it to a gel with PBS or a medium when using it actually. In the present invention, the dry type of collagen-based materials becomes increased in density of collagen fiber tissue by drying to drop out of water from a gel. Even if it is set back to gel with PBS or medium again, it will be small size in volume comparing with original dried collagen-based materials. As a result of this, the dry type of collagen-based materials has a lot of advantage, such as strength and orientation comparing with those of a gel type when manufactured, since an increase in density of the tissue remains.

In such manner, as one feature in the present invention, it is possible to produce a dry type of the collagen-based materials as well as a gel type of the collagen-based materials by setting it back to a gel with PBS or medium.

<Collagen-Binding Site-Containing Growth Factor>

The graft material for nerve regeneration of the present invention can include a “growth factor anchoring type graft material for nerve regeneration” which comprises collagen-based materials containing collagen having an orientation, and in which a collagen-binding site-containing growth factor (hereinafter also referred to as “CB-GF”) comprising a receptor agonist peptide and a collagen-binding peptide binds to the above-described collagen.

In the growth factor anchoring type graft material for nerve regeneration, the nerve regeneration effect based on the collagen-based materials as well as the synergistic nerve regeneration effect based on a growth factor can be expected. Moreover, since the growth factor is bound to collagen fibers of the collagen-based materials, it can stay for a long time at grafted site and promote nerve regeneration persistently.

Although there is no particular restriction on the amount of the CB-GF to be bound to the collagen-based materials, with respect to 1 mg (dry weight) of collagen-based materials a CB-GF is bound in an amount of 0.01 to 1 nmol, preferably 0.1 to 1 nmol, and more preferably 0.5 to 1 nmol. The increasing rate of nerve regeneration is preferable if the CB-GF is bound below 1 nmol; and if the CB-GF is bound beyond 0.01 nmol, the function of a nerve regeneration is more effectively exerted.

(CB-GF)

With respect to a CB-GF, there is no restriction on its structure and production method, insofar as it includes a growth factor receptor agonist peptide (hereinafter, also referred to as a “GF site”) and collagen-binding peptide (hereinafter, also referred to as a “CB site”) and both of the peptides may be bound chemically, or it may be a fusion protein including a GF site and CB site. In this case, for example, the CB site may be binding directly or through a linker composed of a polypeptide fragment with the GF site. Additionally, two polypeptides of the GF site and the CB site may be crosslinked by a reagent including disuccinimidyl glutarate or glutaraldehyde through an amino group. Further, one polypeptide is derivatized by succinimidyl-4-hydrazine nicotinate acetone hydrazone, and the other polypeptide is derivatized by succinimidyl-4-formyl benzoate, and then the two derivatized polypeptides may be mixed for crosslinking through an amino group. Moreover, in addition to the above, the two may be linked by a crosslinking agent other than polypeptides or other compounds to bind the GF site and the CB site.

(Collagen-Binding Peptide)

The “collagen-binding peptide” constituting the CB-GF is a functional site to bind a growth factor receptor agonist peptide to collagen fibers of the collagen-based materials. As described above, although the growth factor exerts a nerve regeneration activity, it may not be expected sustained nerve regeneration activity because of a low local residual ratio by systemic administration such as intravenous injection.

However, by using the CB-GF, the GF site can be bound to the collagen fibers of the collagen-based materials through a CB site contained in the CB-GF, without using a crosslinking agent or other chemical components. The growth factor anchoring type graft material for nerve regeneration can be prepared easily as described below, and is superior in safety since a crosslinking agent is not used.

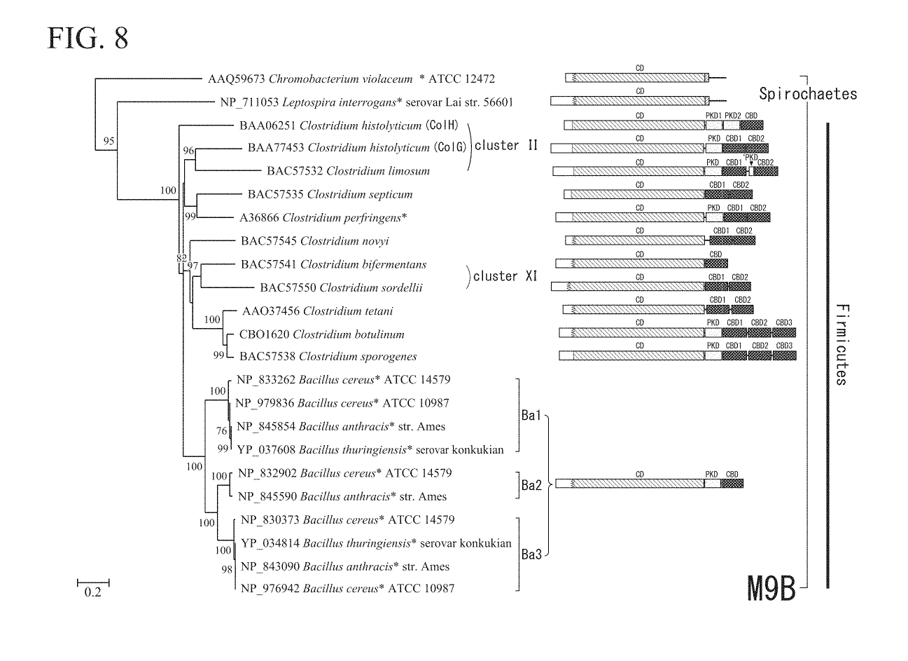

Meanwhile, the “CB site” can include widely what can bind to at least a part of the collagen fibers. Example of a polypeptide bindable to a collagen fiber include a collagenase-derived collagen-binding site. Examples of structural gene for the collagenase-derived collagen binding site include a DNA fragment including a base sequence of base Nos. 3001 to 3366 of a gene (GenBank Accession No. D29981) of Clostridium histolyticum collagenase (hereinafter, occasionally referred to as “Co1H”) set forth in SEQ ID NO: 1. The DNA fragment codes for the amino acid sequence specified by GenBank Accession No. BAA06251, and as shown in FIG. 8, includes a catalytic site represented by CD and a collagen binding site represented by CBD. The amino acid sequence of Nos. 901 to 1021 of an amino acid sequence represented together with the base sequence of SEQ ID NO: 1 corresponds to a CBD. Similarly, Clostridium histolyticum collagenase (hereinafter, occasionally referred to as “Co1G”) specified by GenBank Accession No. BAA77453, Clostridium limosum collagenase specified by ditto BAC57532, Clostridium septicum collagenase specified by ditto BAC57535, Clostridium perfringens collagenase specified by ditto A36866, Clostridium novyi collagenase specified by ditto BAC57545, Clostridium bifermentans collagenase specified by ditto BAC57541, Clostridium sordellii collagenase specified by ditto BAC57550, Clostridium tetani collagenase specified by ditto AAO 37456, Clostridium botulinum collagenase specified by ditto YP_001254122, Clostridium sporogenes collagenase specified by ditto BAC57538, Bacillus cereus collagenase specified by ditto NP_833262, Bacillus cereus collagenase specified by ditto NP_979836, Bacillus cereus collagenase specified by ditto NP_833262, Bacillus cereus collagenase specified by ditto NP_979836, Bacillus anthracis collagenase specified by ditto. NP_845854, Bacillus thuringiensis collagenase specified by ditto YP_037608, Bacillus cereus collagenase specified by ditto NP_832902, Bacillus anthracis collagenase specified by ditto NP_845590, Bacillus cereus collagenase specified by ditto NP_830373, Bacillus thuringiensis collagenase specified by ditto YP_034814, Bacillus anthracis collagenase specified by ditto NP_843090, or Bacillus cereus collagenase specified by ditto NP_976942, and other collagen-binding peptides derived from a bacterial collagenase may be used similarly. Meanwhile, the “CB site” is required to bind to a collagen fiber of the collagen-based materials to extent that the growth factor can be retained there, therefore it is not necessarily contain the entire amino acid sequence of the collagenase-derived collagen binding site. For example, the collagen-binding peptide having 80% or more, 90% or more, 95% or more, or 98% or more homology with a base sequence constituting a CBD in amino acid sequence encoded by a structural gene and capable to bind to a collagen fiber of the collagen-based materials to extent that the growth factor can be retained there may be favorably used. Alternatively, for example, the collagen binding peptide having 80% or more, 90% or more, 95% or more, or 98% or more homology with a base sequence constituting a CBD in amino acid sequence encoded by a structural gene and capable to bind to a collagen fiber of the collagen-based materials to extent that the growth factor can be retained there may be favorably used. There is no particular restriction on binding method, and, for example, it may be bound with affinity for a part of collagen fibers exposing out of a surface of the collagen-based materials. The homology between sequences can be calculated using a known sequence alignment algorithm, Basic Local Alignment Search Tool (BLAST).

(Growth Factor Receptor Agonist Peptide)

A GF site constituting a CB-GF is a site for exerting a function of a growth factor or the like by binding to collagen fibers of collagen-based materials. Examples of a growth factor include an epidermal growth factor (EGF), a nerve growth factor (NGF), a glial cell line-derived neurotrophic factor (GDNF), a fibroblast growth factor (FGF), a platelet-derived growth factor (PDGF), a transforming growth factor beta (TGF-β), an insulin-like growth factor-1 (IGF-1), or a bone morphogenetic protein (BMP), and a growth factor receptor agonist exerting such actions widely may be used. Furthermore, factors such as a brain-derived neurotrophic factor (BDNF), a vascular endothelial growth factor (VEGF) exert a nerve repairing activity, and they can promote nerve regeneration when applied to a defective part.

As a structural gene for such a growth factor receptor agonist, especially use of a basic fibroblast growth factor (bFGF) is preferable. Examples of such a basic fibroblast growth factor include a DNA fragment composed of base sequence of base Nos. 468 to 935 of the Homo sapiens fibroblast growth factor 2 (basic) gene (NCBI Reference Sequence Accession No. NM_002006.4) as set forth in SEQ ID NO: 2. Further, as a structural gene for an epithelial growth factor, there is also cDNA of preproEGF (GenBank Accession No. U04842) of Rattus norvegicus.

As a GF site, a basic fibroblast growth factor (bFGF) may be used favorably in the present invention. Since a basic fibroblast growth factor is superior in nerve regeneration ability, if the CB-GF bound to a basic fibroblast growth factor as a growth factor constituent growth factor (hereinafter, referred to as “CB-bFGF”) is bound to collagen-based materials, nerve can be repaired early. Meanwhile, CB-GF bound to epithelial growth factor (EGF) in place of a basic fibroblast growth factor is referred to as CB-EGF.

Examples of an embodiment of the CB-bFGF include a polypeptide in which the CB is selected from the group consisting of (a) to (c), and the bFGF is a polypeptide selected from the group consisting of (d) to (f):

(a) A polypeptide comprising the amino acid sequence of amino acid Nos. 255 to 375 as set forth in SEQ ID NO: 5

(b) A polypeptide comprising an amino acid sequence wherein 1 to several amino acids are substituted, deleted, inserted or added in the sequence of amino acid Nos. 255 to 375 of the amino acid sequence as set forth in SEQ ID NO: 5, and having binding activity to extent that the growth factor can be retained to a collagen fiber of collagen-based materials

(c) A polypeptide comprising an amino acid sequence having 80% or more sequence identity with the sequence of amino acid Nos. 255 to 375 of the amino acid sequence as set forth in SEQ ID NO: 5 and binding activity to extent that the growth factor can be retained to a collagen fiber of the collagen-based materials

(d) A polypeptide comprising amino acid sequence of Nos. 3 to 157 of the amino acid sequence set forth in SEQ ID NO: 5

(e) A polypeptide comprising an amino acid sequence wherein 1 to several amino acids are substituted, deleted, inserted or added in the amino acid sequence of Nos. 3 to 157 of the amino acid sequence as set forth in SEQ ID NO: 5, and having a nerve repairing activity

(f) A polypeptide comprising an amino acid sequence having 80% or more sequence identity with the amino acid sequence of Nos. 3 to 157 of the amino acid sequence as set forth in SEQ ID NO: 5, and having a nerve repairing activity

(b) In the amino acid sequence in (e), “1 to several” amino acids may be, for example, 1 to 30, 1 to 20, 1 to 10, 1 to 5, or 1 to 3 amino acids.

(c) In the amino acid sequence in (0, the sequence identity of the amino acid sequence is 80% or more and less than 100%, and may be, for example, 85% or more, 90% or more, 95% or more, or 98% or more.

The sequence identity between amino acid sequences can be calculated using a known sequence alignment algorithm, Basic Local Alignment Search Tool (BLAST).

(Linker)

CB-GF may be used what is bound to the CB site and the GF site through a linker.

By insertion of a linker the CB site and the GF site can be isolated by a predetermined gap width, thus each site can independently fully exert each function. As the result, by insertion of the linker, the CB-GF can be bound stronger to collagen fibers than the CB-GF without the linker.

Examples such a linker include a peptide fragment which does not have a specific three-dimensional structure and is composed of amino acids, such as serine, threonine, proline, aspartic acid, glutamic acid, lysine. Further, as such a linker, an amino acid sequence derived from the ColH may be used favorably. More specifically, a polycystic kidney disease I (hereinafter, referred to as “PKD”) domain of ColH may be used favorably. Additionally, a PKD derived from another bacterial collagenase may be also used favorably as the linker. This is because collagen binding activity of the CBD reinforced by coexistence of PKD. Such a linker derived from a bacterial collagenase is depicted in FIG. 8 as PKD. Incidentally, such a linker should preferably be a resistant to a peptide hydrolase or the like contained in a human circulatory liquid, and the local residual performance of the GF site is enhanced and nerve regeneration can be persistently promoted.

<<Method for Producing Graft Material for Nerve Regeneration>>

A method for producing a graft material for nerve regeneration of the present invention comprises a step of immersing collagen-based materials containing collagen having an orientation in a solution containing a collagen-binding site-containing growth factor (CB-GF) comprising a receptor agonist peptide and a collagen-binding peptide, and binding the collagen-binding site-containing growth factor to the collagen.

For example, by adding predetermined amounts of the collagen-based materials containing collagen having an orientation to a phosphate buffer solution, stirring the mixture for 60 seconds to 60 minutes, preferably 5 to 30 minutes, and more preferably 15 to 30 minutes at a temperature 0 to 10° C., or leaving it standing, the CB-GF can be bound to the collagen-based materials.

Since both of the GF site and the CB site constituting CB-GF to be used in the present invention are peptides, they can be prepared as a fusion protein. When the CB-GF includes a basic fibroblast growth factor (bFGF) as a growth factor receptor agonist, and PKD-CBD derived from ColH as a linker and a CB site is herein referred as a bFGF-PKD-CBD, a method for producing the bFGF-PKD-CBD is disclosed in Nishi N. et al.; ProcNatlAcadSci USA vol. 95, pages 7018-7023, 1998. The bFGF-PKD-CBD can be produced by this method. Further by using a basic fibroblast growth factor (bFGF) as a GF site, and a CBD derived from ColG as a CB part, bFGF-CBD can be also produced by fusing these. By using a gene sequence for epithelial cell growth factor (EGF) instead of gene sequence for bFGF, a CB-EGF can be produced similarly as above. Further by using a gene sequence coding for an another growth factor receptor agonist, a CB-GF in which the another growth factor receptor agonist bind to the CB can be produced. Meanwhile, as described above, the CB site and the GF site may be crosslinked by a crosslinking agent.

<<Kit for Producing Graft Material for Nerve Regeneration>>

A kit for producing a graft material for nerve regeneration of the present invention comprises collagen-based materials containing collagen having an orientation, and a collagen-binding site-containing growth factor (CB-GF) comprising a receptor agonist peptide and a collagen-binding peptide.

Examples of a graft material for nerve regeneration include the material described in <<graft material for nerve regeneration>> above. The CB-GF may be in such a state of a CB-GF solution containing the CB-GF. Examples of the CB-GF solution include a solution dissolving CB-GF in a buffer solution in a range of 0.5 to 2.0 mg/ml.

Examples of buffer solutions include a phosphate buffer solution of pH 7.0 to 8.0, Tris buffer solution, and a physiological saline solution. Since components necessary for producing a growth factor anchoring type graft material for nerve regeneration are included in the kit of the present invention, the growth factor anchoring type graft material for nerve regeneration can be easily produced.

<<Nerve Regeneration Method, Etc.>>

The graft material for nerve regeneration of the present invention described in <<graft material for nerve regeneration>> above can be used for nerve regeneration. Further transplanting the graft material for nerve regeneration to a treatment target can be carried out as a nerve regeneration method.

In one embodiment, the present invention provides a graft material comprising collagen-based materials containing collagen having an orientation for nerve regeneration.

In one embodiment, the present invention provides a use of a graft material comprising collagen-based materials containing collagen having an orientation for nerve regeneration.

In one embodiment, the present invention provides a method for regenerating a nerve, including transplanting a graft material comprising collagen-based materials containing collagen having an orientation to a patient or an affected animal necessitating a treatment.

Examples of transplantation include a method such as filling a nerve defect region, crosslinking a nerve defect region, coating a nerve defect region, filling a nerve damage region, crosslinking a nerve damage region and coating a nerve damage region. For example, mention may be made of a method of transplanting a graft material for nerve regeneration having a length approximately equal to the length of a nerve defect region to a nerve defect region of a patient or an affected animal.

There is no particular restriction on a type of nerve applied, and it is possible to apply to nerves such as a central nerve, peripheral nerve, motor nerve, sensory nerve, etc.

Nerve regeneration should show at least one of various phenomena occurring in a course of a nerve repair or a nerve generation such as an increase, proliferation and maturation of cells. Further, as the result, the nerve regeneration may preferably include a phenomenon that an original nerve function can be recovered fully or partially.

Whether effective nerve regeneration was accomplished or not can be confirmed by a known method. For example, if a patient or an affected animal in which a nerve damage was occurred and a graft material was transplanted have higher extent of recovery of nerve function comparing with a patient or affected animal in which a nerve damage was occurred and a graft material was not transplanted, it can be determined that effective nerve regeneration was accomplished. The recovery of the nerve function can be evaluated by a response to stimulation and a recovery of a motor function as a standard, as described in the following examples.

Nerve may be regenerated by cells (endogenous cells) derived from cells in a defect region and originally existing in a region for a treatment, or for example, also by cells (exogenous cells) transplanted with a graft material for nerve regeneration. As these cells, a mention may be made nerve cells, neural precursor cells, embryonic stem cells, artificial pluripotent stem cells, mesenchymal stem cells, vascular endothelial cells, vascular endothelial progenitor cells, hematopoietic stem cells etc.

### Examples

Next, the present invention will be described in further detail with reference to Examples, but the present invention is not limited to the Examples below.

### [Production of Oriented Collagen Tube]

First, according to the method disclosed in Patent Literature 4, an oriented collagen tube A comprised of collagen-based materials containing collagen having an orientation and having characteristics described below was produced.

Raw collagen: Porcine Skin Collagen type I (Manufacturer: Nippi, Specifications: Pepsin solubilized, 10 mg/mL, 20 mM acetic acid, 0.8 μm filtered)

Shape of collagen-based materials: Cylinder shape, 7-layered, inner-seamless cylinder,

Thickness of collagen-based materials: about 15 μm (1 layer, dry), about 105 μm (7 layers, dry)

Inner diameter: 1 mm,

Collagen orientation: a long axes direction (1 to 7 layers),

Amount of collagen (7 layers, dry): about 25 mg/cm².

A specific method for producing a collagen tube A is as follows.

First of all, an oriented collagen gel with a string shape was prepared. As to a collagen gel, 10 mg/mL of type I collagen solution derived from a porcine skin (by Nippi) was extruded through a nozzle having 0.38 mm of an inner diameter into a plate containing 10-fold diluted phosphate-buffered saline (10×PBS) at 38° C. at pH 7.4, and thereby sliding the nozzle to obtain a string shape collagen gel having about 1 mm in diameter and about 200 mm of length.

The orientation of the obtained collagen gel was analyzed by Raman spectroscopy (PHOTON Design Corporation). In doing so, an excitation wavelength was set at 514.5 nm using a continuous oscillation argon ion laser Stabilite 2017 (Spectra-Physics Inc.), and HR-320 (Jovin Yvon S.A.S.) as a spectrometer and LN/CCD-1100-PB/UV AR/1 (Roper Scientific, Inc.) as a detector were used respectively. As the result of analysis, it was recognized that collagen fibers were orientated to a long axes direction of the collagen gel.

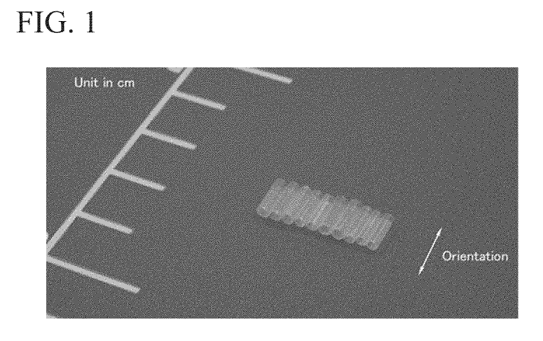

The produced oriented collagen gel with a string shape was aligned on an axle rod to an axes direction, and after that was dried to obtain an oriented collagen material with a tube shape. Further, the alignment of the produced oriented collagen gel with a string shape on the dried oriented collagen material with tube shape was repeated to obtain 7 layers. After that the axle rod was removed to obtain a dried oriented collagen tube A (FIG. 1).

The collagen tube A consisted of 7-layered collagen-based materials. A collagen tube A′ consisting of 3-layered collagen-based materials was produced under the same conditions as described above, except that the collagen-based materials were stacked in 3 layers. The collagen tube A′ has the following characteristics.

Shape of collagen-based materials: Cylinder shape, 3-layered inner-seamless cylinder,

Thickness of collagen-based materials: about 15 μm (1 layer, dry), about 45 μm (3 layers, dry),

Amount of collagen (3 layers, dry): about 11 mg/cm².

(Raw collagen, the inner diameter, and orientation of the collagen (1 to 3 layers) are the same as that of the collagen tube A)

[Production of bFGF-PKD-CBD Fusion Protein]

A bFGF-PKD-CBD fusion protein was produced according to the method disclosed in International Publication No. 2012/157339.

A concrete production method of the bFGF-PKD-CBD fusion protein is as follows.

Firstly, a DNA fragment (PKD-CBD gene) including the sequence of base Nos. 2719 to 3391 of Co1H gene set forth in SEQ ID NO: 1 was inserted into a SmaI site of a pGEX-4T-2 plasmid (by GE Healthcare, Japan) in the usual manner. Meanwhile, a DNA fragment (bFGF gene) consisting of a base sequence of base Nos. 468 to 932 in Homo sapiens fibroblast growth factor 2 (basic) gene (NCBI Reference Sequence Accession No. NM_002006.4) set forth in SEQ ID NO: 2 was amplified by a PCR method so as to have a BglII site at the 5′ end, and one nucleotide (G residue) and an EcoRI site at the 3′ end. The amplified DNA fragment (bFGF gene) was inserted into a BamHI-EcoRI site of the plasmid inserted the DNA fragment (PKD-CBD gene) in the usual manner, thereby preparing an expression plasmid. The obtained expression plasmid possesses a reading frame (SEQ ID NO: 4) coding a GST-bFGF-PKD-CBD fusion protein (SEQ ID NO: 3). The amino acid sequence of the bFGF-PKD-CBD fusion protein is set forth in SEQ ID NO: 5, and the base sequence of the bFGF-PKD-CBD fusion protein is set forth in SEQ ID NO: 6. In the amino acid sequence set forth in SEQ ID NO: 5, the N-terminal 2 amino acid residues Gly-Ser are a part of a recognition site of a GST tag cleavage enzyme (thrombin protease). The expression plasmid was introduced into Esherichia coli BL21 Codon Plus RIL (by Stratagene) by electroporation method to produce a transformant.

The transformants were precultured overnight in 50 mL of 2×YT-G culture medium containing 50 μg/ml of ampicillin and 30 μg/ml of chloramphenicol. Ten mL of the obtained preculture solution was added to 500 ml of the culture medium and shake-cultured at 37° C. until the turbidity (O.D. 600) of the resulting bacterial suspension reached approximately 0.7. To the obtained bacterial suspension 5 mL of 0.1 M isopropyl-β-D-thiogalactopyranoside (IPTG) solution was added and the mixture was cultured at 25° C. for 5 hours. After adding 5 mL of isopropanol solution to 0.1 M phenylmethylsulfonyl fluoride (PMSF), the bacterial suspension was centrifuged at 6000×g and 4° C. for 10 minutes to collect the transformant. The transformant was suspended in 7.5 mL of 50 mM Tris-HCl (pH 7.5), 5M NaCl and 1 mM PMSF, and the cells were disrupted by a French press. To 19 volumes of the suspension, 1 volume of 20% Triton (registered tradename) X-100 was added and stirred at 4° C. for 30 minutes. The obtained bacterial suspension was centrifuged at 15,000×g and 4° C. for 30 minutes, and the supernatant was recovered. The obtained supernatant was further centrifuged again at 15,000×g and 4° C. for 30 minutes, and the suspension was recovered. The supernatant was defined as a clarified lysate. The clarified lysate was added to 2 mL of glutathione-sepharose beads, and stirred at 4° C. for 1 hour. After washing the beads 5 times with 12 mL of 50 mM Tris-HCl (pH 7.5) and 0.5 M NaCl, the beads were suspended in a small amount of 50 mM Tris-HCl (pH 7.5) and 0.5 M NaCl, and filled in a column, and then the GST-bFGF-PKD-CBD fusion protein was eluted therefrom with an elution liquid (50 mM Tris-HCl (pH 8.0), 0.5 M NaCl, 10 mM glutathione). Thrombin in amount of 5 units per 1 mg of the fusion protein was added and allowed to react at 25° C. for 10 hours. The obtained reaction solution was added to 1 mL of heparin-sepharose beads and stirred at 4° C. for 3 hours allowing the bFGF-PKD-CBD fusion protein to bind to the beads. After discarding the supernatant gently, the beads were washed three times with 12 mL of 50 mM Tris-HCl (pH 7.5) and 0.5 M NaCl. The beads were filled in a column, and the protein was eluted with 10 mL of 50 mM Tris-HCl (pH 7.5) with salt gradient of NaCl from 0.5 to 2 M, to obtain the bFGF-PKD-CBD fusion protein (SEQ ID NO: 5).

### [Transplantation Test 1-1]

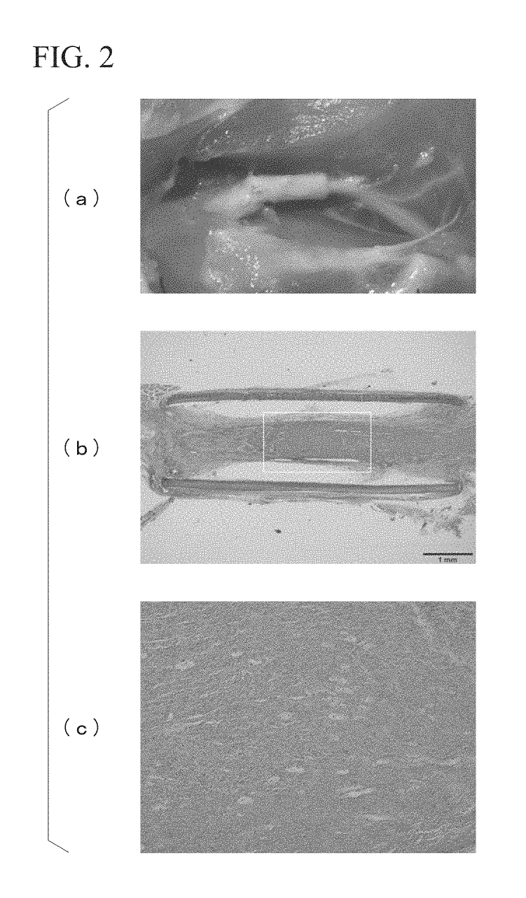

The sciatic nerve of a 7-week-old Wistar rat was cut to 5 mm. An oriented collagen tube A with 5 mm length was transplanted into the defective part and crosslinked. FIG. 2 shows the transplanted part 12 weeks after the transplantation. Nerve regeneration was recognized in the oriented collagen tube A (FIG. 2).

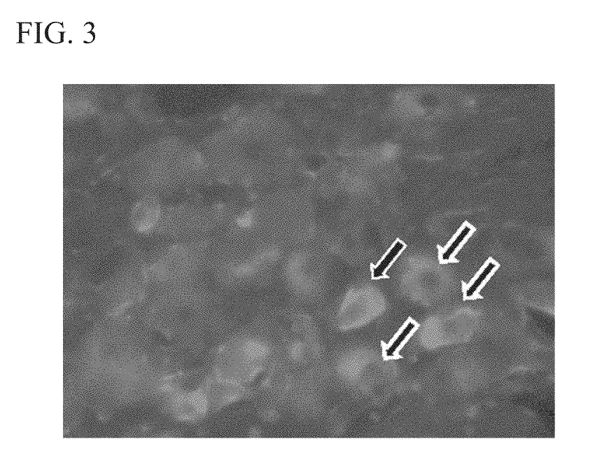

A retrograde neuronal tracer (Fast Blue) was administered to the rat and L5 dorsal root ganglion cells after the nerve regeneration was observed. The result is shown in FIG. 3. L5 dorsal root ganglion cells stained with Fast Blue were detected (arrow in the drawing), and it was shown that the nerve after the regeneration was functional.

### [Transplantation Test 1-2]

A transplantation test was carried out similarly as described in [Transplantation test 1-1], except that a collagen tube A′ was used instead of the collagen tube A.

When the collagen tube A′ was used, collagen-based materials of the collagen tube were occasionally split during transplantation. After transplantation nerve regeneration in the collagen tube A′ was recognized.

Therefore, the collagen tube A was superior to the collagen tube A′ in terms of transplantation efficiency and nerve regeneration efficiency.

### [Transplantation Test 2]

Firstly, the oriented collagen tube A was produced as described above.

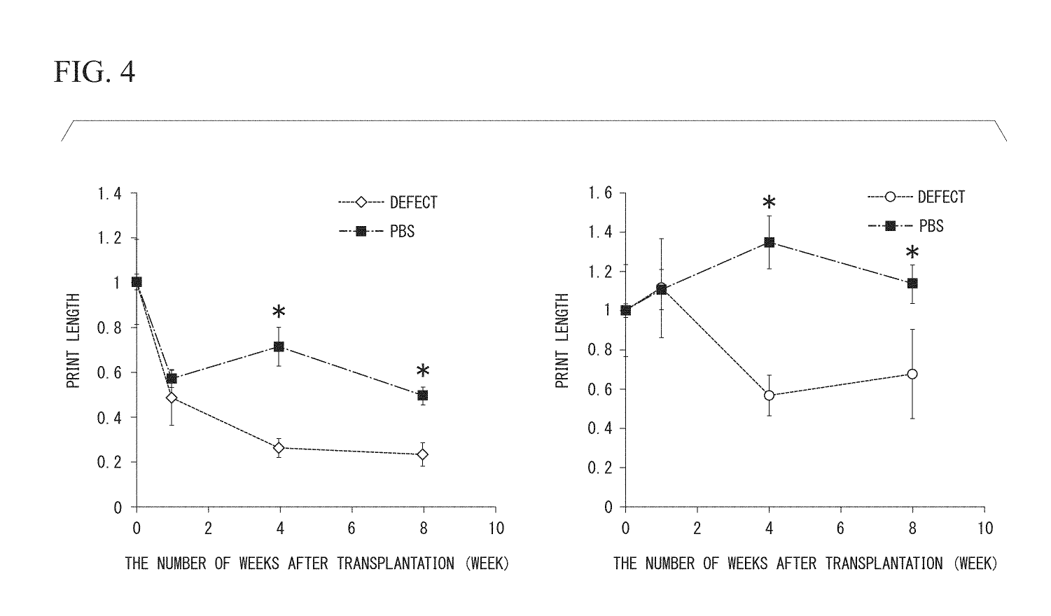

Sixteen 7-weeks-old Wistar rats were provided for the test. The rats were divided into two groups: a group in which 15 mm of the sciatic nerve is defected to extent that natural healing cannot generally recognized (defective group); and a group in which an oriented collagen tube A was immersed in phosphate buffer and after that it was transplanted into the 15 mm-defected part of the sciatic nerve and then the detective part was crosslinked with the 15 mm collagen tube (PBS group; n=8). One, four and eight weeks after the transplantation, print width and print length of the foot sole using a rat walking analyzer (CatWalk). A value before the defect is set as 1, and the evaluation result is shown in FIG. 4.

Referring to FIG. 4, print width in the PBS group was significantly larger than that in the defective group. Further print length in the PBS group was also significantly longer than that in the defective group and equivalent to the print length before the defect.

From these result, it was revealed that the extent of recovery of a motor function in the PBS group is superior to that in the defective group. Accordingly, it was shown that the oriented collagen tube A has an excellent nerve regeneration effect.

### [Production of Growth Factor Anchoring Type Oriented Collagen Tube and CB-GF Binding Test]

A solution in which a bFGF-PKD-CBD fusion protein was dissolved in phosphate buffer at a concentration of 1.25, 2.5, 5, or 10 mg/ml was prepared, and the oriented collagen tube was added to the solution.

The binding amount of the bFGF-PKD-CBD fusion protein to the oriented collagen tube was determined from the amount of the bFGF-PKD-CBD fusion protein in a supernatant solution as follows.

Binding amount=Addition amount−Amount of bFGF-PKD-CBD fusion protein in a supernatant solution

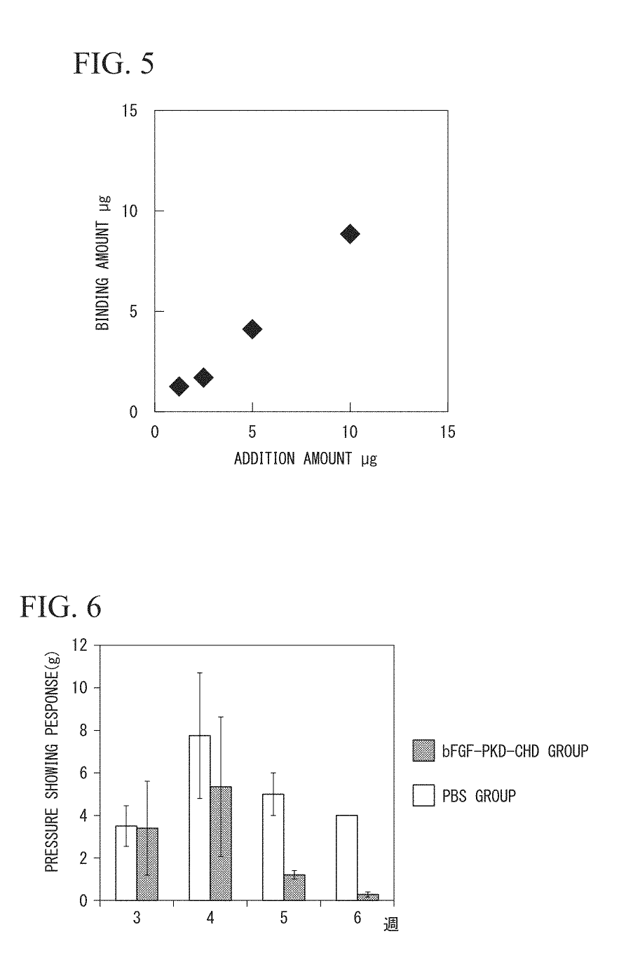

A result of the binding test is shown in FIG. 5. The graph of FIG. 5 shows the relationship between the addition amount of the bFGF-PKD-CBD fusion protein and the binding amount of the bFGF-PKD-CBD fusion protein to the oriented collagen tube. When 10 μg of the bFGF-PKD-CBD fusion protein was added, about 9 μg of the fusion protein of that was bound. From the graph of FIG. 5, it was found that even when an other amount of the fusion protein was added, approximately 90% of the protein binding rate was achieved. From these results, it was shown that bFGF can be highly effectively anchored to an oriented collagen tube by the bFGF-PKD-CBD fusion protein, and that a growth factor anchoring type oriented collagen tube was obtained.

### [Transplantation Test 3]

Firstly, as described above, the oriented collagen tube A was immersed in 10 mg/ml of a bFGF-PKD-CBD solution to produce a growth factor anchoring type oriented collagen tube (oriented collagen tube B).

Eight 7-weeks-old Wistar rats were provided for the test. The rats were divided into two groups: a group in which the oriented collagen tube A was immersed in phosphate buffer and after that was transplanted (PBS group); and a group in which the growth factor anchoring type oriented collagen tube B was transplanted (bFGF-PKD-CBD group). To each rat, 15 mm of a sciatic nerve was defected to extent that a natural healing cannot generally recognized, and then the defected part was crosslinked with each collagen tube with a length of 15 mm.

From two weeks after the transplantation, behavioral evaluation using von Frey filament was carried out, and recovery of the sensory nerve was evaluated. In the behavioral evaluation, the ratio of rats responding to 0.008 to 300 g of plantar stimuli and an average value of a threshold at which the rats responded were calculated. The evaluation was carried out at week 2, 3, 4, 5, and 6 after the transplantation. The evaluation results are shown in Table 1, and FIG. 6.

TABLE 1
Sensory nerve recovery rate of rats

| | Week after transplantation | | | | |
|---|---|---|---|---|---|
| | 2 Week | 3 Week | 4 Week | 5 Week | 6 Week |
| bFGF-PKD-CBD group | 4/4 | 4/4 | 4/4 | 2/2 | 2/2 |
| PBS group | 2/4 | 4/4 | 4/4 | 2/2 | 2/2 |

Table 1 shows the ratio of sensory nerve recovery (the number of recovered individuals/the number of evaluated subjects) of the rats. The recovery evaluation was evaluated by the presence or absence of the responses to 300 g of plantar stimuli. Sensory nerve recovery was recognized in both of the PBS group and the bFGF-PKD-CBD group. Therefore, it was shown that a nerve defect which extent is usually difficult to heal by a natural healing can be regenerated with both of the oriented collagen tube A and the oriented collagen tube B.

While the PBS group showed sensory nerve recovery in all cases (4 out of the 4 cases) at week 3 after the transplantation, the bFGF-PKD-CBD group showed sensory nerve recovery in all cases at 2 weeks after the transplantation. From this, it was revealed that the bFGF-PKD-CBD group showed a regeneration of a sensory nerve earlier than the PBS group.

Referring to FIG. 6, it is indicated that the bFGF-PKD-CBD group responded to a lower stimulus (pressure) than the PBS group.

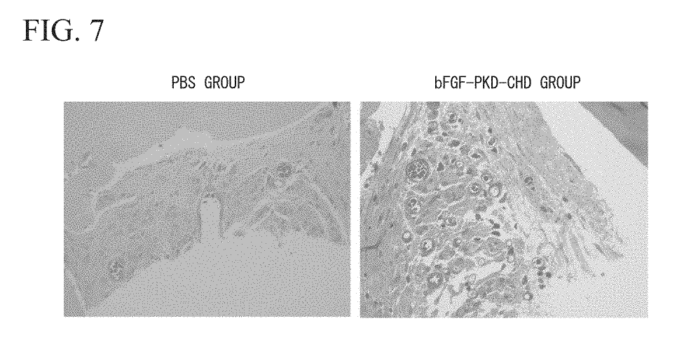

Further, FIG. 7 shows a state of the regenerated nerve. FIG. 7 shows a toluidine blue-stained image of the nerve regenerated 8 weeks after the transplantation of the collagen tube. The bFGF-PKD-CBD group showed a higher level of myelination than the PBS group.

From these result, it was revealed that the bFGF-PKD-CBD group is functionally and histologically superior to the PBS group in quality of the recovery of a regenerated nerve.

Configurations and combinations thereof in the embodiments described above are merely examples, and thus addition, deletion, substitution and other modification of the configuration are possible without departing from the spirit of the present invention. Further, the present invention is not limited to each embodiment, but only to the scope of the claims.

### INDUSTRIAL APPLICABILITY

According to the present invention, a graft material for nerve regeneration capable of effectively regenerating nerve is provided.

1-10. (canceled)

11. A graft material for nerve regeneration, comprising:
collagen-based materials containing collagen having an orientation,
wherein a collagen-binding site-containing growth factor comprising a receptor agonist peptide and a collagen-binding peptide binds to the collagen, and the receptor agonist peptide and the collagen-binding peptide are linked by a linker; and
the linker is a polycystic kidney I domain of collagenase.

12. The graft material for nerve regeneration according to claim 11, having a hollow cylindrical shape and in which at least a part of the inner surface of the cylinder is formed of the collagen-based materials.

13. The graft material for nerve regeneration according to claim 12, wherein the collagen-based materials have a cylindrical shape and are a seamless tube without a junction.

14. The graft material for nerve regeneration according to claim 12, wherein collagen has an orientation in a direction in which openings at both ends of the cylinder are connected.

15. The graft material for nerve regeneration according to claim 11, wherein the growth factor receptor agonist peptide is a basic fibroblast growth factor.

16. The graft material for nerve regeneration according to claim 11, wherein the collagen-based materials are comprised of a plurality of collagen-based materials layers.

17. The graft material for nerve regeneration according to claim 11, wherein a thickness of the collagen-based materials is 50 μm or more and 200 μm or less.

18. A method for producing a graft material for nerve regeneration, comprising a step of
immersing collagen-based materials containing collagen having an orientation in a solution containing a collagen-binding site-containing growth factor comprising a receptor agonist peptide and a collagen-binding peptide, and binding the collagen-binding site-growth factor to the collagen.

19. A kit for producing graft material for nerve regeneration, comprising:
collagen-based materials containing collagen having an orientation; and
a collagen-binding site-containing growth factor comprising a receptor agonist peptide and a collagen-binding peptide.
